(19) United States
(12) Patent Application Publication
(10) Pub. No.: US 2010/0089271 A1
(43) Pub. Date: Apr. 15, 2010

(54) NOVEL EXPLOSIVES

(76) Inventors: Martin E. Colclough, Kent (GB); Peter J. Haskins, Kent (GB); Ross W. Millar, Kent (GB); Ian C. Sage, Worcestershire (GB)

Correspondence Address: NIXON & VANDERHYE, PC, 901 NORTH GLEBE ROAD, 11TH FLOOR, ARLINGTON, VA, 22203, US

(21) Appl. No.: 12/527,030

(22) PCT Filed: Feb. 18, 2008
(86) PCT No.: PCT/GB08/00547

§ 371 Date: Aug. 13, 2009

(30) Foreign Application Priority Data
Feb. 19, 2007 (GB) 0703065.3

(51) Int. Cl. F42B 12/20; C07C 69/76; C07C 291/08; C06B 25/04; F42C 14/00; F42C 99/00

(52) U.S. Cl. 102/473; 560/85; 534/566; 149/105

(57) ABSTRACT

This invention relates to the field of novel explosives, compositions and munitions comprising the same, as well as methods of synthesis and their use. The invention particularly relates to the field of explosives which are switchable between two different liquid crystalline states, and which possesses a general formula below.

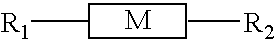

wherein M is a mesogenic core, R₁ and R₂ are independently selected terminal end groups attached to the mesogenic core, and wherein the mesogenic core contains sufficient (NO₂)ₓ to provide an explosive output. The explosive compound may form part of an explosive composition, which may be used as a high explosive fill for any part of a munition. The explosive composition may contain other components such as non-explosive LC compounds, or binders, or further known explosive compounds. The explosive composition has one or more liquid crystalline states, which may possess differing sensitivities

This invention relates to the field of explosives and especially to the field of high explosives. The present invention concerns novel explosive compounds, compositions and munitions comprising the same, as well as methods of synthesis and their use.

By the term “munition” as used hereinafter is meant a bomb, warhead or rocket, shell or any similar device which contains a high explosive.

The present invention is particularly concerned with the provision of high explosives for use in munitions. The storage of such explosives is hazardous due to their inherent sensitivities. There have been a number of disasters over the last 40 years, involving ships, magazines and weapon storage depots resulting in loss of life and military equipment. The present invention is concerned with the development of explosives that are tailored with novel functionality.

According to a first aspect of the invention there is provided the use of a liquid crystalline compound comprising a nitrated mesogenic core group, having two terminal end groups attached thereto, to form an explosive, wherein the compound is capable of switching between at least two liquid crystalline states.

In a second aspect there is provided the use of a liquid crystalline compound comprising a nitrated mesogenic core group, having two terminal end groups attached thereto, as a high explosive, wherein the sensitivity of the explosive is capable of being changed by the application of at least one stimulus.

According to one aspect of the present invention, there is provided an explosive compound of Formula (I),

R₁—(R₁₇—W₁)_{f}—(R₁₈—W₂)_{g}—(R₁₉)—R₂ Formula (I)

where R₁ and R₂ are each independently selected from hydrogen, a functional group, cyano, nitro, halo, optionally substituted hydrocarbyl, optionally substituted alkoxy, optionally substituted heterocyclyl, any of which may be optionally interposed with one or more oxygen or sulphur atoms,
where R₁₇, R₁₈ and R₁₉ are each independently selected from cycloalkyl, aryl or heterocyclic rings, any of which may be optionally substituted by one or more groups selected from nitro, halo, optionally substituted hydrocarbyl or cyano; where
W₁ and W₂ are independently selected from, a direct bond, —C(O)O—, —OC(O)—, —CH₂—, —CH₂CH₂—, —CH₂O—, —OCH₂—, —O—, —S—, —N═N(O)—, —N═N—, —CH═N—, —CH═CH—, or —C≡C—;
and where f is 1 or 2, g is 0, 1 or 2, provided that f+g is less than 3,
provided that each of R₁₇, R₁₈ and R₁₉, when present, is substituted by one or more nitro groups.

In yet a further aspect there is provided the use of the above compound as an explosive, and in particular, its use in accordance with the first and second aspects as specified above.

Preferably, at least one of R₁ and R₂ is other than hydrogen or nitro, more preferably both R₁ and R₂ are selected from a group other than nitro, further to improve the liquid crystal switching properties of the compound.

Preferably the explosive is a high explosive, capable of sustaining detonation. It is desirable to produce novel explosives which are capable of switching between at least two liquid crystalline states. These different crystalline states may lead to different physical or chemical properties in the different states, such as, for example the different liquid crystalline states may each possess a different level of sensitivity.

Preferably W₁ and W₂ are selected from; a direct bond, —N═N(O)—, —N═N—, —C(O)O— or —OC(O)—. Further, when the linkage is selected as azoxy (—N═N(O)—) or azo (—N═N—), the adjoining rings will preferably be in the trans configuration. Preferably, the connections W₁ and W₂ between adjacent rings are para i.e. to give a linear configuration, as linear rod-like structures enhance the LC switching ability.

Preferably the linking groups are flexible such as, for example, ester, azoxy or azo. These groups permit facile synthesis and increased LC properties on the system. A yet further advantage is their stability, both to temperature and to synthesis reagents. Preferably, W₁ and W₂ are independently selected from, —N═N(O)—, —C(O)O— or —OC(O)— or a combination thereof.

Preferably R₁₇, R₁₈ and R₁₉ are independently selected from phenyl, naphthalene, 1,3 benzodioxanes, pyrimidine, pyridine, piperidine; furan, thiophene, oxazole, thiazole, oxadiazole, 1,3,4-thiadiazole bicyclo(2.2.2)octane, cyclohexane, dioxane, and may be optionally substituted with nitro, F, Cl, Br or CN and may be present in any of the available substitution positions. Preferably R₁₇, R₁₈ and R₁₉ are six-membered rings.

Typically R₁₇, and R₁₉ (terminal rings), each independently comprise 1, 2 or 3 nitro groups and R₁₈ (as a non-terminal ring) comprises, 1 or 2 nitro groups, more preferably substituted by at least 2 nitro groups. Even more preferably R₁₇, R₁₈, and R₁₉ are selected from phenyl, substituted by at least one nitro group, more preferably 2 nitro groups. The nitro groups that are present on the R₁₇, R₁₈, and R₁₉ rings are preferably not in a terminal end group position, i.e. do not form part of R₁ or R₂.

It is theoretically possible to have 4 or even 5 nitro groups on a 6-membered ring system, however ring deactivation due to the presence of at least one nitro group does not readily allow for such a high degree of nitration. Clearly, there is a practical limit to the number of nitro groups that may be placed on any given ring. Conveniently, such as, for example, on a 6-membered ring, a ring in a terminal position may have 1, 2 or 3 nitro groups present and when the ring is adjoined to two other rings then the ring may have 1 or 2 nitro groups present. Clearly, naphthyl or ring systems with greater than 6 atoms, such as, for example, fused cyclic ring systems, may be able to possess more nitro groups than a 6-membered ring system.

The terminal end groups R₁ and R₂ may be any group which facilitates the liquid crystal switching effect. Preferably R₁ and R₂ may be independently selected from halo, cyano, a functional group, optionally substituted hydrocarbyl, or an optionally substituted heterocyclyl; any of which may be optionally interposed with one or more oxygen or sulphur atoms and may be substituted with one or more nitro or nitrate ester groups. It is desirable to include one or more nitro or nitrate ester groups as substituents onto the moiety which forms the terminal end group, to increase the energy of detonation of the compound.

In a preferred embodiment at least one of R₁ and R₂ are each selected from an optionally substituted hydrocarbyl, which may be optionally interposed with one or more oxygen or sulphur atoms. More preferably at least one of R₁ and R₂ are selected from branched or a straight chain alkyl, alkoxy, alkenyl, alkenyloxy, alkanoyloxy, alkenoyloxy; any of which may be optionally substituted with nitrate ester, nitro or halo.

Even more preferably R₁ or R₂ may be independently selected from branched or a straight chain alkyl or alkoxy and contain 1 to 20 carbon atoms which may be optionally interposed with one or more oxygen or sulphur atoms and may optionally be substituted with nitro or nitrate ester. It is desirable to include one or more nitro or nitrate ester groups onto the hydrocarbyl chains, to increase the energy of detonation of the compound. There will be fewer nitro groups present on the mesogenic core when at least one of R¹ and R² is selected from optionally substituted hydrocarbyl, which will reduce the detonation energy of the overall compound. It is therefore desirable to substitute nitro or nitrate ester groups onto the hydrocarbyl terminal end group, to increase the detonation energy of the compound.

LC properties are displayed in more suitable (i.e. lower) temperature ranges when flexible alkyl or alkoxy chains are attached to one of the terminal aryl groups in the para-position. R₁ and R₂ are attached in a -1,4- i.e. para position with respect to the terminus ring.

In an alternative arrangement one of the terminal end groups may be a pendant group on a polymer backbone, such that the compound of Formula I is side-chain in a polymeric liquid crystal material.

According to a further aspect of the invention there is provided an explosive compound comprising a nitrated mesogenic core group, and two independently selected terminal end groups attached to said mesogenic core to provide an explosive compound capable of switching between at least two liquid crystalline states.

The degree of nitration on the compound needs to be sufficient to allow an explosive output to be achieved. It is preferable that at every ring which is present in the mesogenic core group possess at least one nitro group, more preferably each ring possess at least two nitro groups.

A simple schematic representation of said explosive may be that of formula II below, wherein M is a mesogenic core, R₁ and R₂ are independently selected terminal end groups attached to the mesogenic core, and wherein the mesogenic core contains sufficient (NO₂)ₓ to provide an explosive output, typically where x is at least 2, preferably at least 4, more preferably at least 6.

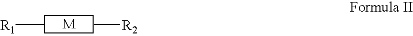

In certain configurations it may be desirable to include further high energy groups, such as, for example, nitro or nitrate ester groups onto the terminal end chains. Preferably, at least one of the terminal end groups comprises at least one nitro or nitrate ester group. Preferably there are at least two nitro or nitrate ester groups present on at least one of the terminal end groups.

There is further provided a method of preparing an explosive compound capable of switching between at least two liquid crystalline states, comprising the step of providing a nitrated mesogenic core group with two independently selected terminal end groups attached to said mesogenic core group.

There is further provided the use of a compound comprising a nitrated mesogenic core group, having two independently selected terminal end groups attached thereto, so as to form an explosive compound capable of switching between at least two liquid crystalline states. Such use will usually involve forming an explosive, such as a high explosive or, indeed, and explosive device.

There is further provided the use of a compound comprising a nitrated mesogenic core group, having two independently selected terminal end groups attached thereto, as a high explosive, wherein the sensitivity of the explosive is designed so as to be capable of being changed by the application of at least one stimulus.

In a further aspect of the invention, there is provided an explosive liquid crystalline compound, comprising a nitrated mesogenic core group, having two independently selected terminal end groups attached thereto, of general Formula (I),

R₁—(R₁₇—W₁)_{f}—(R₁₈—W₂)_{g}—(R₁₉)—R₂ (I)

where R₁ and R₂ are each independently selected from hydrogen, a functional group, nitro, cyano, halo, optionally substituted hydrocarbyl, optionally substituted alkoxy, optionally substituted heterocyclyl, any of which may be optionally interposed with one or more oxygen or sulphur atoms;
R₁₇, R₁₈ and R₁₉ are each independently selected from cycloalkyl, aryl or heterocyclic rings, any of which may be optionally substituted by one or more groups selected from nitro, halo, optionally substituted hydrocarbyl or cyano; where
W₁ and W₂ are independently selected from, a direct bond, —C(O)O—, —OC(O)—, —CH₂—, —CH₂CH₂—, —CH₂O—, —OCH₂—, —O—, —S—, —N═N(O)—, —N═N—, —CH═N—, —CH═CH—, or —C≡C—;
and where f is 1 or 2, g is 0, 1 or 2, provided that f+g is less than 3,
provided that each of R₁₇, R₁₈ and R₁₉, when present, is substituted by one or more nitro groups.

The invention relates in particular to explosives that can be made to alter their sensitivity in response to controlled stimuli. Hence our preferred embodiment involves switchable explosives i.e. explosives which can have their crystalline state altered by the application of a stimulus, their methods of manufacture and devices incorporating said compounds.

Organic compounds which are capable of forming solid crystalline structures are typically able to exist in one or more different solid crystalline states. Many of the military explosives that are in current use are aromatic (organic) compounds some of which may possess more than one solid crystalline state. Typically, some of the solid crystalline states are more sensitive to stimuli than other crystalline states. A change in solid crystalline state can only be brought about by solvent recrystallisation to form a different solid crystalline state, requiring the controlled evaporation of solvent, such as, for example, controlled temperatures and/or pressures. Clearly recrystallisation between different solid crystalline state is not a workable solution to provide a means of changing or switching the sensitivity of an explosive compound in a munition. Liquid crystal compounds, mixtures and the corresponding LCD devices are well known. The phrase “liquid crystal” refers to compound(s) which, as a result of their structure, have a phase or phases intermediate between liquid and solid and which are characterised by orientational ordering and a decrease in positional ordering, usually at working temperatures for example, of from ±40 to 200° C. Liquid crystals can exist in various phases. Compounds which exhibit liquid crystal properties find utility by their ability to align themselves and to change their alignment under the influence of voltage, particularly in liquid crystal displays, to alter the path of polarised light. The alignment of the compounds may also be changed by other stimuli, such as magnetic or thermal stimuli.

For a fuller description of liquid crystal phases and devices see for example “The Handbook of Liquid Crystals”, Ed D Demus, J Goodby, G W Gray, H-W Spiess, V Vill, Pub WileyVCH, 1998.

Compounds which exhibit liquid crystal properties have the ability to adopt more than one liquid crystalline state. The present inventors unexpectedly found that the use of a plurality of nitro groups on a mesogenic core of a liquid crystalline type compound may be used to provide a switchable explosive compound, exhibiting two or more liquid crystalline states, these states may possess different sensitivities to particular stimuli in their different states. Compounds of Formula I exhibit explosive behaviour in at least one crystalline state, but may be capable of switching between at least two crystalline states, and may possess differing sensitivity.

Preferably there are sufficient nitro groups present on the mesogenic core and/or as optional substituents on the terminal end groups such that a high order event, either deflagration or detonation can occur. The higher the degree of nitration on the compounds of Formula (I), the higher the energy of detonation of the molecule, i.e. the more energy will be released when the molecule undergoes deflagration or detonation.

Clearly there is a limit to the number of nitro groups that may be placed on a given ring. In the case of aromatic phenyl rings this is due to the deactivating nature of the nitro group on the aromatic ring.

The terminal end group affects the polarisabilty of a liquid crystal material i.e. controls the extent of switching of the compound. It is well known in the art that at least one of the terminal end groups is preferably selected from a rod like group, typically a hydrocarbyl chain. Therefore to increase the LC character of compounds of Formula (I) it is desirable to select terminal end groups which enhance the LC effect.

The mesogenic core is the basic structural unit of a polymer having the requisite anisotropic shape and attractive interactions to establish long range intermolecular order in its liquid phase. It is this feature which provides the liquid crystal with its liquid crystalline properties. A definition of the term “mesogenic” and other related liquid crystal terminology can be found in an article prepared by IUPAC published in Pure Appl. Chem., Vol. 74, No. 3, pp. 493-509, 2002.

The mesogenic core may be linear or bent. Preferably, the mesogenic core will be linear, such as, for example, 6-membered rings linked in a -1,4- i.e. (para) manner.

The mesogenic group will usually have 2, 3 or 4 rings in the system, depending on their respective sizes; typically 3 ring systems provide optimum switching properties. However 4 rings may be expected to provide higher output energies due to the ability of increasing the number of possible nitro groups that may be incorporated in the compound.

As used herein, the term “hydrocarbyl” refers to any structure comprising carbon and hydrogen atoms. For example, these may be alkyl, alkenyl, alkynyl, aryl such as phenyl or naphthyl, arylalkyl, cycloalkyl, cycloalkenyl or cycloalkynyl. Suitably they will contain up to 20 and preferably up to 10 carbon atoms.

The term “heterocyclic” includes aromatic or non-aromatic rings, for example containing from 4 to 20, suitably from 5 to 10 ring atoms, at least one of which is a heteroatom such as oxygen, sulphur or nitrogen. Examples of such groups include furyl, thienyl, pyrrolyl, pyrrolidinyl, imidazolyl, triazolyl, thiazolyl, tetrazolyl, oxazolyl, isoxazolyl, pyrazolyl, pyridyl, pyrimidinyl, pyrazinyl, pyridazinyl, triazinyl, quinolinyl, isoquinolinyl, quinoxalinyl, benzthiazolyl, benzoxazolyl, benzothienyl or benzofuryl.

As used herein, the term “alkyl” refers to straight or branched chain alkyl groups, suitably containing up to 20 and preferably up to 6 carbon atoms, and the term “alkoxy” relates to ±0-alkyl groups. The term “alkenyl” and “alkynyl” refer to unsaturated straight or branched chains which include for example from 2-20 carbon atoms, for example from 2 to 6 carbon atoms. In addition, the term “aryl” refers to aromatic groups such as phenyl or naphthyl. The terms “cycloalkyl”, “cycloalkenyl” and “cycloalkynyl” refer to such groups which are cyclic and have at least 3 and suitably from 5 to 20 ring atoms. These rings may be fused together to form bicyclic, tricyclic or even larger multiple ring systems.

Optionally substituted hydrocarbyl groups may be substituted by functional groups, or by other types of hydrocarbyl group or nitro or nitrate ester groups. For example, cyclic groups such as aryl, heterocyclic or cycloalkyl, cycloalkenyl or cycloalkynyl, any of which may be substituted by hydrocarbyl chains such as alkyl, alkenyl or alkynyl groups as well as functional groups. Where the hydrocarbyl group is itself an alkyl, alkenyl or alkynyl group, it may be substituted with cyclic groups such as heterocyclic groups, aryl groups, cycloalkyl, cycloalkenyl or cycloalkynyl groups, as described above, which may themselves be further substituted by hydrocarbyl or functional groups. Optionally substituted hydrocarbyl may also have one or more non-adjacent carbon atoms replaced by O, S, C(O)O, or OCO or —C≡C—. Preferably hydrocarbyl groups have one or more non-adjacent carbon atoms replaced by O or S and may be optionally substituted by nitro, nitrate ester or halo.

The term “functional group” refers to reactive groups such as, for example, oxo, C(O)OR^{a}, C(O)R^{a}, OC(O)R^{a}, OR^{a}, S(O)ₜR^{a}, NR^{b}R^{c}, OC(O)NR^{b}R^{c}, C(O)NR^{b}R^{C}, —NR^{b}C(O)OR^{a}, —NR^{b}C(O)R^{a}, —NR^{a}CONR^{b}R^{c}, ═NOR^{a}, —N═CR^{b}R^{c}, S(O)ₜNR^{b}R^{c} or —NR^{b}S(O)ₜR^{a} where R^{a}, R^{b} and R^{c} are independently selected from hydrogen or optionally substituted hydrocarbyl, or R^{b} and R^{c} together form an optionally substituted ring which optionally contains further heteroatoms such as sulphur, S(O), S(O)₂, oxygen and nitrogen, t is 0 or an integer of from 1-3.

The term “heteroatom” as used herein refers to non-carbon atoms such as oxygen, nitrogen or sulphur atoms as mentioned above. Where the nitrogen atoms are present, they may be present as part of an amino residue such that they will be substituted for example by hydrogen or hydrocarbyl, preferably hydrogen or alkyl. In a further aspect of the invention there is provided a method of producing explosive liquid crystal compounds by nitrating a commercially available LC compound or mixture using any known nitration technique to provide a polynitrated LC compound.

An explosive compound is one which undergoes a rapid reaction when subjected to a stimulus, typically heat or shock. The rate of the reaction determines whether the explosive event is a fast burn, such as provided by pyrotechnic or propellants, or a high order event such as deflagration or detonation, as provided by high explosives. Typically, compounds of Formula I may be used as high explosives which undergo detonation upon the action of an initiator.

According to a further aspect of the invention there is provided an explosive composition comprising at least one compound according to Formula (I), which composition is formulated for use as a high explosive, propellant or the like. High explosives are also used in initiators or booster charges. There may be two or more compounds of Formula (I) present in an explosive composition.

Compounds of Formula (I) may be mixed with known explosive compounds or explosive formulations that do not have liquid crystalline character, this may further increase the output energy of the explosive composition.

According to a yet further aspect there is provided an explosive composition comprising at least one compound according to Formula (I) or explosive composition and at least one non-explosive liquid crystal compound or non-explosive liquid crystal mixture. It may be advantageous to add non-explosive liquid crystal compounds or mixtures to help with the LC switching behaviour of the at least one compound of Formula (I) or explosive composition. A non-explosive LC compound or mixture is one which does not undergo an explosive event such as for example, deflagration or detonation.

In a further aspect the explosive composition may comprise at least one compound according to Formula (I), optionally at least one non-explosive liquid crystal compound or non-explosive liquid crystal mixture, and optionally at least one known explosive compound or explosive formulation that does not have liquid crystalline character.

It may be desirable for the explosive composition to comprise at least one non-explosive liquid crystal material, which may improve the switching ability of the explosive composition. By non-explosive LC material, we mean LC compounds that do not possess sufficient energy within the compound to sustain a high order reaction.

In LC devices, mixtures are commonly used to achieve desirable switching properties. It would be clear that an excessive amount of non-explosive LC material will adversely affect the ability of the compounds of Formula (I) to sustain an explosive or high order event, such as, for example, detonation. Therefore the at least one non-explosive liquid crystal compound or liquid crystal mixture may be present in the range of from 0.5% to 20%, preferably 0.5% to 5% by volume of the explosive material.

In one arrangement, the compound of Formula I may be pressed or cast into a munition casing. In such a use, said explosive composition may additionally comprise a binder to aid consolidation or mixing. The binder may be selected from a polymer or non-metal salt, such as for example a metal stearate, waxes, PTFE, polyethylene or epoxy resins. In a preferable aspect the polymer is an energetic polymer. The energetic polymer is selected from Polyglyn (Glycidyl nitrate polymer), GAP (Glycidyl azide polymer) or Polynimmo (3-nitratomethyl-3-methyloxetane polymer). The binder may be present in a total amount of from 0.5% to 20%, preferably 0.5% to 5% by volume of the explosive material. Where both an additional non-explosive liquid crystal material or mixture and a binder are present then they will be present in a total amount of less than 20%, preferably in the range of from 0.5% to 5% by volume of the explosive material.

According to a further aspect there is provided a munition device comprising at least one compound of Formula (I) or an explosive composition as hereinbefore defined. Conveniently the munition may have part, substantially all, or all of its explosive content present as an explosive composition as hereinbefore defined. A munition may be a bomb, warhead or rocket, shell or any similar device which contains an explosive material, such as for example a high explosive.

According to a further aspect of the invention there is provided a munition comprising an activation means which is capable in use of causing a change in the sensitivity of a compound of Formula (I) or an explosive composition, as hereinbefore defined. Preferably the stimulus may be an electromagnetic field (EMF) stimulus, such as, for example, IR, light, microwaves, an electric field, or magnetic field. Alternative stimuli may be an electric voltage or heating. The stimulus causes a change in the crystalline state to part of or substantially all of a compound of Formula (I) or an explosive composition as hereinbefore defined. Preferably, the stimulus is an applied electrical voltage or induced current. The stimulus which causes the change in crystalline state may not be provided by the initiator, which is capable of initiating the explosive in the munition. Preferably the stimulus is selected such that in use it is not able to cause detonation of the compounds of Formula I.

In a typical liquid crystal device, such as, for example a monitor or display device, there is typically provided a device comprising two spaced cell walls each bearing electrode structures and treated on at least one facing surface with an alignment layer, a layer of a liquid crystal material or mixture enclosed between the cell walls. The electrodes are driven by a voltage, which causes a change in the alignment of the liquid crystalline material.

Therefore in one arrangement according to the invention, a voltage may be applied directly by the use of contact electrodes spaced over the surface of the explosive composition or via a conductive body which is in intimate contact with said explosive composition. Alternatively electric field and/or magnetic field may be generated by coils placed in close proximity to the explosive compound or composition to bring about the change in sensitivity. Typically, there will have to be two distinct voltage planes, across which the potential difference is applied.

The change in temperature may preferably be by the application of heat, this may be provided by a heating means, such as, for example, electrical elements, flame, or chemical heating, such as, for example a pyrotechnic for example thermite.

In a preferred embodiment a munition comprises an activation means comprising at least one stimulus, which is capable in use of causing a change in the sensitivity of a compound of Formula (I), or an explosive composition, as hereinbefore defined. Preferably the at least one stimulus is an electromagnetic field (EMF), applied electric voltage, induced current, magnetic field or heating.

According to a yet further aspect of the invention there is provided a method of changing the sensitivity of a compound of Formula (I) or an explosive composition, comprising the steps of applying a stimulus to part of or substantially all of said compound or composition.

There is further provided the use of a compound of Formula (I) or an explosive composition as a high explosive fill for a munition, which has one or more liquid crystalline states, wherein at least one liquid crystalline state has decreased sensitivity with respect to the other state. The high explosive may form part of the main charge or part of the explosive train, or it may form part of a safety and arming unit (SAU).

According to a yet further aspect of the invention there is provided the use of a compound of Formula (I) or an explosive composition as a high explosive fill for a munition, wherein the sensitivity is capable of being changed by the application of a stimulus.

Liquid crystalline compounds will switch back to their original (or relaxed state) when the stimulus is removed. The sensitivity of compounds of Formula (I) or explosive compositions as hereinbefore defined may be switched to its original state by the removal of said stimulus.

In liquid crystal display devices, it is an essential feature of the liquid crystal material that the switching time from one liquid crystalline state to another liquid crystalline state is in the range of a few milliseconds. This allows the picture or image to be updated without any time lag. The switching time for compounds of Formula I, when used in a munition may not require such rapid switching rate.

In an alternative embodiment further liquid crystalline states may be achieved by the application of a different stimulus or an increased level of the original stimulus. It may be desirable to use one or more stimuli to cause switching of the liquid crystalline state of a compound of Formula (I) or explosive composition, as this may avoid accidental switching.

There is further provided a munition comprising a compound of Formula (I) or an explosive composition, as hereinbefore defined, wherein the munition is so arranged that external heating (such as accidental heating) of the munition will cause the explosive composition to switch to a less sensitive crystalline state. This may function as part of a passive mitigation system.

The change is sensitivity may be from a more sensitive state to a less sensitive state or from a less sensitive state to a more sensitive state. These states may be determined by the addition of other materials, such as those as defined hereinbefore.

Particular preferred aryl species are detailed below and specific preferred examples of suitable compounds of Formula (I) are set out in Tables 1-34

### Trinitrobenzoates

Particular examples of trinitrobenzoates are compounds of Formula III, where R₁ and R₂ are as hereinbefore defined and R₃ and R₄ are independently selected from hydrogen, nitro, nitrate ester, halo or cyano, and preferably from nitro or hydrogen. The linkages W₁ and W₂ are both esters. The rings at the termini of the mesogenic core may possess up to 3 nitro substituents, which is the maximum nitration pattern available on a phenyl ring.

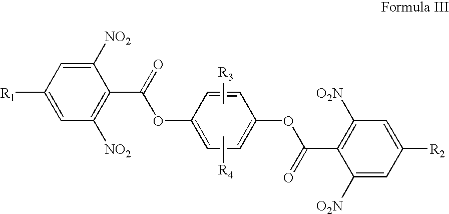

### Dinitrobenzoates

Compounds of Formula V are dinitrobenzoates, where R₁, R₂ are terminal end groups as hereinbefore defined and are both other than nitro R₃ and R₄ are as hereinbefore defined, provided that at least one of R₃ or R₄ on each ring is nitro. The linkages W₁ and W₂ are both selected as ester linkages.

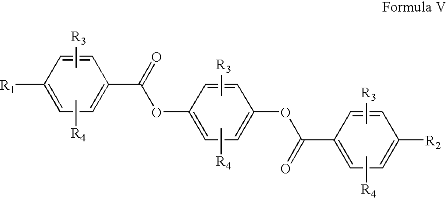

Similarly Formula IV which is a particular example of Formula V, shows a preferred nitration pattern. Formula IV shows R₁ and R₂ are both selected as hydrogen and R₃ and R₄ may be independently selected from hydrogen or nitro.

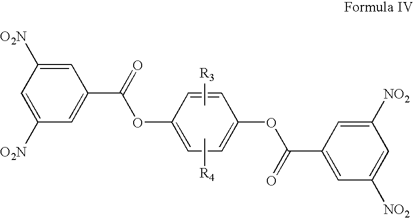

Formula IV shows the nitration pattern when only two nitro groups are present on the terminal rings.

Further examples of compounds of Formula (V), which have the nitration pattern on the terminal rings fixed, are compounds of Formula (X) and are set out in Table 1.

TABLE 1
compounds of Formula (X)

Formula X

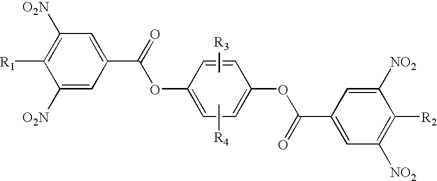

| R³ | R⁴ | R¹ | R² |
|---|---|---|---|
| H | NO₂ | H | —O(CH₂)₄ONO₂ |
| NO₂ | NO₂ | H | —O(CH₂)₄ONO₂ |
| H | NO₂ | H | —O(CH₂)₂O(CH₂)₂ONO₂ |
| NO₂ | NO₂ | H | —O(CH₂)₂O(CH₂)₂ONO₂ |
| H | NO₂ | H | —O(CH₂)₂OCH₂C(NO₂)₃ |
| NO₂ | NO₂ | H | —O(CH₂)₂OCH₂C(NO₂)₃ |
| H | NO₂ | H | —O(CH₂)₂OCH₂ OCH₂C(NO₂)₃ |
| NO₂ | NO₂ | H | —O(CH₂)₂OCH₂ OCH₂C(NO₂)₃ |

The terminal end groups, in Table 1, which contain three nitro groups on one carbon atom will be more stable than their di-nitro counterparts. To improve the stability of terminal end groups possessing di-nitro compounds, the di-nitro groups may be placed on a non terminal carbon atom.

### Azoxy Compounds

Further examples of compounds of Formula (I) are compounds of Formula (XIV) where R₁, R₂, R₃ and R₄ are as hereinbefore defined, W₁ and W₂ linkages are both selected from azoxy and have a trans relationship, such that the two adjoining phenyl groups are in trans relationship.

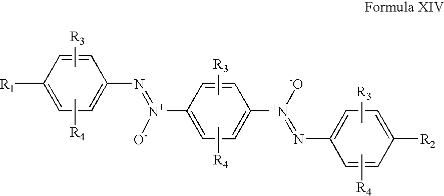

In a particular example, a tri-nitrated ring may be present providing a compound of Formula (XV), where terminal end groups R₁ and R₂ are both nitro and R₃ and R₄ on the central ring may be independently selected from hydrogen or nitro.

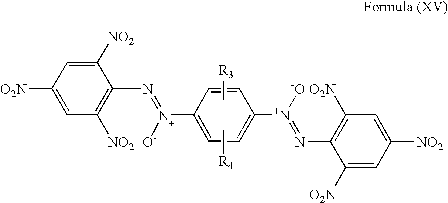

### Mixed Azoxy/Ester Linkage

Examples of compounds of Formula (I) which contain mixed linkages are compounds of Formula (XIX), where R₁, R₂, R₃ and R₄ are as hereinbefore defined, and W₁ is an azoxy and W₂ is an ester.

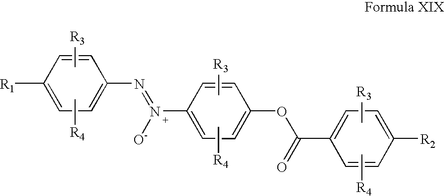

Particular examples of Formula (XIX), are those with a di-nitration pattern and form compounds of Formula (XX), and are present in Table 2. Compounds of Formula (XX) comprise an azoxy linkage and an ester linkage.

TABLE 2
compounds of Formula (XX)

Formula (XX)

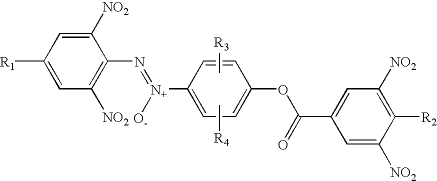

| | | | |
|---|---|---|---|
| R³ | R⁴ | R¹ | R² |

| | | | |
|---|---|---|---|
| H | H | NO₂ | H |
| H | NO₂ | NO₂ | —O(CH₂)₄ONO₂ |
| NO₂ | NO₂ | NO₂ | —O(CH₂)₄ONO₂ |
| H | NO₂ | NO₂ | —O(CH₂)₂O(CH₂)₂ONO₂ |
| NO₂ | NO₂ | NO₂ | —O(CH₂)₂O(CH₂)₂ONO₂ |
| H | NO₂ | NO₂ | —O(CH₂)₂OCH₂C(NO₂)₃ |
| NO₂ | NO₂ | NO₂ | —O(CH₂)₂OCH₂C(NO₂)₃ |
| H | NO₂ | NO₂ | —O(CH₂)₂OCH₂OCH₂C(NO₂)₃ |
| NO₂ | NO₂ | NO₂ | —O(CH₂)₂OCH₂OCH₂C(NO₂)₃ |

### Azo and Ester Linkages

Particular examples of compounds of Formula (I) which have azo and ester linkages are compounds of Formula (XXIV) where R₁, R₂, R₃ and R₄ are as hereinbefore defined, and W₁ is an azo and W₂ is an ester.

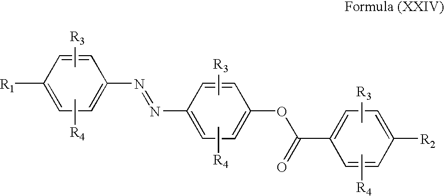

Particular examples of compounds of Formula (XXIV), are those with a di-nitration pattern and form compounds of Formula (XXV), and are presented in Table 3.

TABLE 3
Compounds of Formula (XXV)

Formula (XXV)

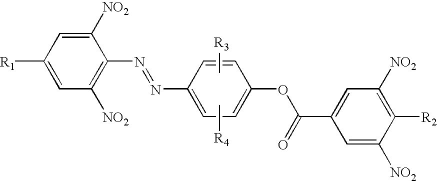

| R³ | R⁴ | R¹ | R² |
|---|---|---|---|
| H | H | NO₂ | H |
| H | NO₂ | NO₂ | —O(CH₂)₄ONO₂ |
| NO₂ | NO₂ | NO₂ | —O(CH₂)₄ONO₂ |
| H | NO₂ | NO₂ | —O(CH₂)₂O(CH₂)₂ONO₂ |
| NO₂ | NO₂ | NO₂ | —O(CH₂)₂O(CH₂)₂ONO₂ |
| H | NO₂ | NO₂ | —O(CH₂)₂OCH₂C(NO₂)₃ |
| NO₂ | NO₂ | NO₂ | —O(CH₂)₂OCH₂C(NO₂)₃ |
| H | NO₂ | NO₂ | —O(CH₂)₂OCH₂OCH₂C(NO₂)₃ |
| NO₂ | NO₂ | NO₂ | —O(CH₂)₂OCH₂OCH₂C(NO₂)₃ |

### Tetracyclic Ring Systems

Particular examples of compounds of Formula (I) are compounds of Formula (XXX) where R₁, R₂, R₃ and R₄ are as hereinbefore defined. The linkages are all selected from azoxy, in a trans configuration.

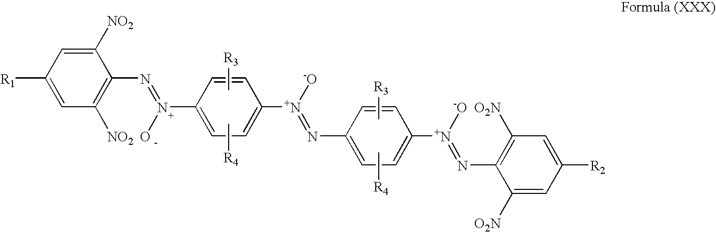

In order to impart more energy into the compounds of Formula I it is desirable to introduce nitro groups or nitrate ester groups into the compound. The examples provided above, demonstrate that it is possible to introduce nitro groups onto the rings or nitro and/or nitrate ester groups on the terminal end groups. It may be desirable to further increase the energy of the compounds of Formula I by introducing energetic groups onto the linking groups.

The azoxy and azo linkage have the advantage of providing additional energy compared to linking groups which contain only carbon, hydrogen and oxygen. The azoxy linkage provides additional energy from both the nitrogen content and the energetic oxygen, N—O functional group. The azo linkage derives extra energy only from the nitrogen. The azo and azoxy linkages are more energetic than the esters, or covalent bonds described above. This is because a preformed CO₂ or covalent bond, such as, for example, a direct bond —C═C— or —C≡C-moiety does not contribute to the overall energetic output. However, the ester functionality may provide a more facile synthesis route which may outweigh the slight difference in energy of a particular compound.

A further advantage of the azoxy and azo linkages over ester linkages, is that compounds comprising tri-substituted aromatic rings are more stable when there is an azo or azoxy linkage present. Hence picryl derivatives, which are unstable, may be considered as potential candidate compounds to be stabilised by the use of adjacent azo or azoxy linkages. The picryl moiety shown below is structurally very similar to TNT (trinitrotoluene); this will help to provide compounds with a very high energy of combustion/detonation.

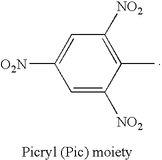

According to a second aspect of the invention there is provided any known process for preparing a compound of Formula (I) as hereinbefore defined.

The nitration reactions which are described below may be carried out by any known method of nitration, such as, for example by mixed acids such as for example a mix of nitric and sulphuric acids, or by using DNPO as described in WO90/01028 or by using umpolung reagents as described in WO97/22590 The constituent parts may be nitrated either before or after they are coupled/reacted together to form a compound of Formula (I).

### Reaction Schemes

By way of example only, the following reaction schemes are proposed for the synthesis of some examples of compounds of Formula (I). The trinitrobenzoate esters may be prepared by the following Scheme 1.

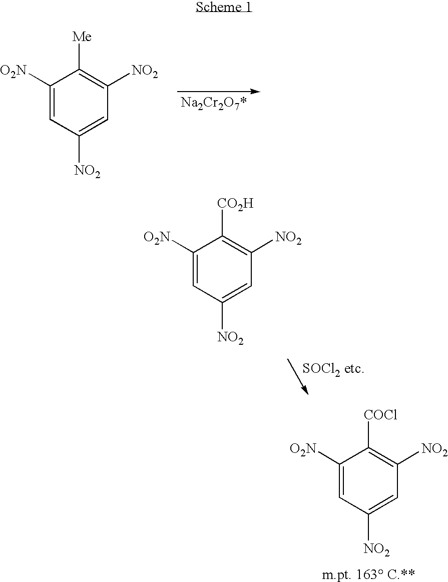

Trinitrotoluene can be oxidised to its corresponding carboxylic acid by known means, such as, for example, using sodium chromate. (* Ref. Vogel, Textbook of Practical Organic Chemistry, 2nd Ed., 719, Longmans, 1951.) The carboxylic acid may then be taken to the corresponding acid chloride by any suitable means, such as, for example, thionyl chloride, to produce a known compound trinitrobenzoyl chloride, (**Ref. E J Fendler J Org. Chem 36 1544 1971).

The trinitrobenzoyl chloride may then be reacted with an alcohol under basic conditions to produce a corresponding ester by the elimination of HCl. In scheme 2, two aliquots of trinitrobenzoyl chloride are reacted with one aliquot of the diol, p-hydroxyphenol (hydroquinone), to form the corresponding diester which forms a compound of Formula (IIIa), where R₃ and R₄ are hydrogen.

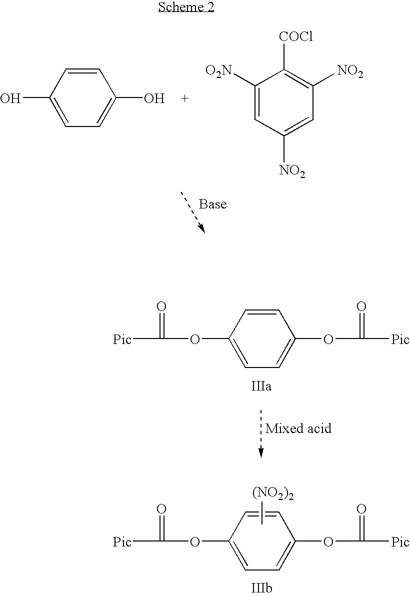

The above formed diester compound of Formula (IIIa) may then be subjected to any known nitration method to add either one or two nitro groups to the 1,4-phenylene moiety, such that a compound of Formula (IIIb) is formed, where R₁, R₂, R₃ and R₄ are all nitro groups.

The dinitrobenzoate esters, scheme 3, may be synthesised in the same manner as described for the trinitrobenzoate esters. The nitration step may be carried out as hereinbefore described and may be controlled to produce either the mono or di-nitro1,4-phenylene moiety of a compound of Formula (IVa) where R₁ and R₂, are both hydrogen and R₃ and R₄ are both nitro.

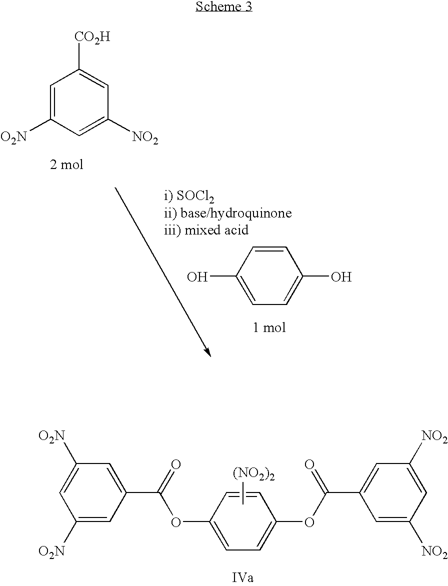

### Mixed Dinitrobenzoates

Scheme 4 shows an example of forming dinitrobenzoates with one or more terminal end groups.

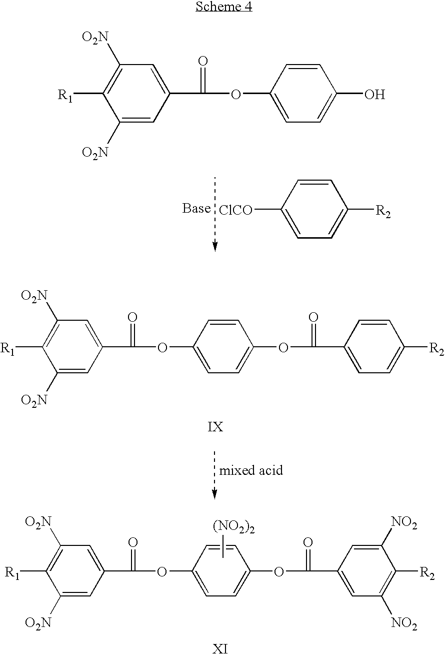

The mono ester bearing the R₁ substituent, may be prepared using one aliquot of both an R₁-substituted acid chloride and hydroquinone, similar to that shown in scheme 3. The mono ester may then be further reacted with a further R₂-substituted benzoyl chloride, which may be the same or different acid chloride as the first acid chloride. To increase the LC behaviour of the molecule preferably the groups R₁ and/or R₂ will be in the para-position. The substituent may be any one of the groups hereinbefore defined in relation to R₁ and/or R₂. Compounds of Formula (IX) may be nitrated as hereinbefore defined to provide a compound as defined in Formula (X).

Alternatively, if a trinitrobenzoate ester is required such that either R₁ or R₂ is selected as nitro then a picryl derivative may be used as shown in scheme 1 and scheme 2.

Conveniently if the R₁ and/or R₂ terminal end chain possess hydroxyl groups they may be elaborated to the corresponding nitrate ester functionality during the final nitration step, such as for example as illustrated in scheme 5 below.

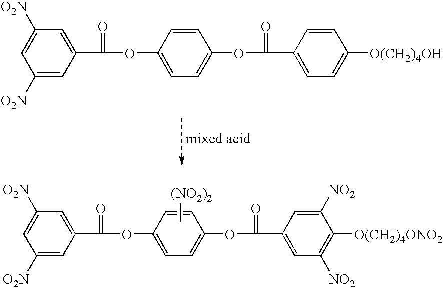

### Azoxy Linked Compounds

A convenient synthesis of the azoxy linkage is to react a substituted nitro phenyl with a (para)di-nitrated phenyl in the presence of sodium arsenite under basic conditions. The remaining nitro group on the phenyl ring may be further elaborated with a second substituted nitro phenyl under the same conditions.

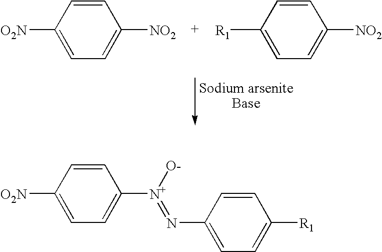

In order to provide sufficient chemical energy to the mesogenic core the rings must be partially nitrated, providing a compound of Formula (XIV) as hereinbefore defined.

### Azoxy Ester

It may be desirable to introduce at least one azoxy bond to a compound of Formula (I), to increase the overall energy.

In scheme 5, H. Zollinger, “Azo & Diazo” Chemistry, Aliphatic & Aromatic Compounds”, p. 192, Interscience, 1961, have shown the synthesis when R₁ is hydrogen.

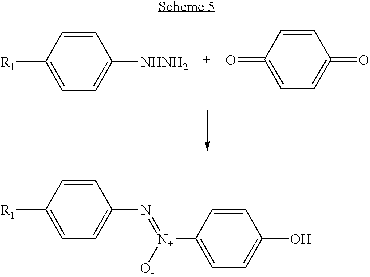

Clearly the resulting phenolic oxygen may be elaborated to an ester functionality with a corresponding substituted carboxylic acid or substituted acid chloride. The above schemes 1 to 5, serve as an indication as to how certain preferred linkages may be introduced between phenyl ring structures.

The methods of providing linkages between rings may be applied to heterocyclic and/or non-aromatic ring systems. The other linkages defined in relation to W₁ and/or W₂ may be produced using known methods which would be apparent to the skilled chemist, such as for example, —C═C— and —C≡C— may be easily formed using catalysed coupling reactions, such as, for example Stille or Suzuki coupling.

Liquid crystal materials are useful, in particular, in display devices where their ability to align themselves and to change their alignment under the influence of voltage, is used to impact on the path of polarised light, thus giving rise to liquid crystal displays. These are widely used in devices such as watches, calculators, display boards or hoardings, televisions and computer screens, in particular, laptop computer screens etc. The properties of the compounds which impact on the speed with which the compounds respond to voltage charges include molecule size, conductivity, viscosity, dielectric anisotropy (Δ∈) or dipole moment and in the smectic C phase the spontaneous polarisation.

According to a further aspect of the invention there is provided a liquid crystal mixture comprising at least one compound of Formula (I) as hereinbefore defined. The compounds of the invention may also prove useful as dopants for use in liquid crystal mixtures.

Accordingly there is further provided a liquid crystal device comprising at least one compound of Formula (I) or a liquid crystal mixture as hereinbefore defined.

There is yet further provided the use of a compound of Formula (I) as a liquid crystal compound and additionally a method of using a compound of Formula (I) in a liquid crystal display device.

The present invention is further illustrated, by way of example only, in the following experimental Examples.

### EXPERIMENT 1

### Nitration of 4-(pentyloxy)-4-Biphenyl Carbononitrile

4-(Pentyloxy)-4-biphenyl carbononitrile (0.3 g, 1.13 mmol) was added to white fuming nitric acid (5 ml, 98%) in a ice-cooled glass beaker (10 ml) at 0-5° C. with stirring. On each addition a transient blue color was noticeable which rapidly disappeared upon stirring.

After the addition (2-3 min.) the yellow/brown solution was allowed to warm up to room temperature and stirred for an additional 2-3 hr, before adding rapidly to ice (100 g). The off white material was filtered under suction and washed with water (100 ml) and allowed to dry in a vacuum oven at 50° C. overnight to give a fine off-white powder (0.34 g).

Recrystallization of approximately 0.2 g of material from ethanol gave a light yellow crystalline solid (0.14 g) M.P. 127° C.

¹H and ¹³C nmr experiments (CDCl₃) indicated a trinitrated product 2 with the structure shown below. The IR was also consistent with a nitrated product.

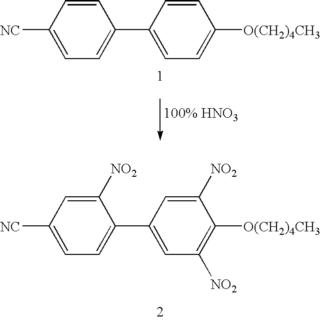

### ¹³C NMR:

| δ/ppm | Assignment |
|---|---|
| 14.3 | A |
| 22.7 | B |
| 27.9 | C |
| 29.9 | D |
| 79.1 | E |
| 128.7 | H |
| 133.6 | K |
| 136.7 | L |
| 132.0 | M |
| 129.2 | N |
| 116.1 | P |

### ¹H NMR:

| δ/ppm | Integration | Multiplicity | Assignment |
|---|---|---|---|
| 0.97 | 3 | Triplet | A |
| 1.47 | 4 | Multiplet | B + C |
| 1.89 | 2 | Multiplet | D |
| 4.26 | 2 | Triplet | E |
| 7.98 | 2 | Singlet | H |
| 7.65 | 1 | Doublet | K |
| 8.05 | 1 | Multiplet | L |
| 8.42 | 1 | Singlet | N |

### Structure of Compound 2

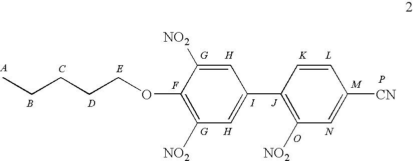

### Infra-Red

1526, 1546 (str., —NO₂); 2233 (med., —CN).

### EXPERIMENT 2

### Physical Properties Study of Nitrated Compound 2

Six mixtures containing varying percentages of compound 2 and compound 1 (3 mixtures containing each compound) in the commercially available nematic mixture ZLI-1132 (Merck, Darmstadt) were prepared and the clearing points of the mixtures together with that of the undoped ZLI-1132 host were determined by polarizing optical microscopy using a heated stage. The transition was quite broad, and the initial appearance of the nematic phase on cooling was taken for calculation purposes. An extrapolation of the data to 100% concentration of the compound under test provides an approximation of the transition temperatures of compound 2 and compound 1.

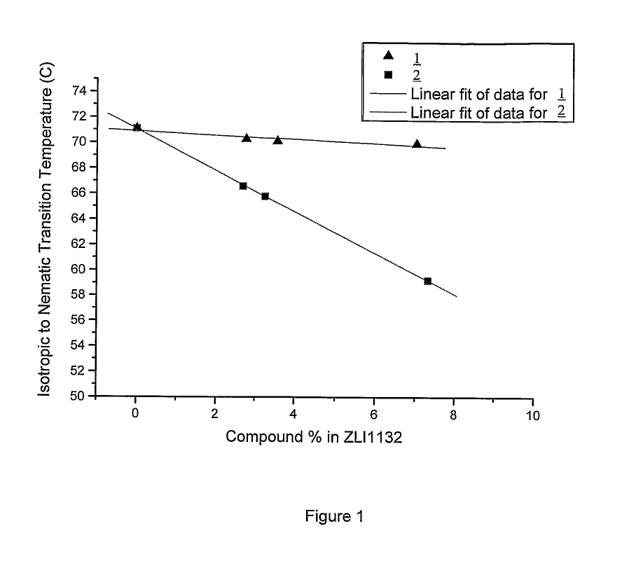

FIG. 1: shows the extrapolated nematic to isotropic transition temperatures

Compound 1: 54.1° C. (literature value of 67.0° C.)

### Compound 2: −90.9° C.

In a further experiment a mixture of 25% of compound 2 and 75% of compound 1 was prepared; this composition did not exhibit a nematic phase above room temperature or the recrystallisation temperature of the mixture. On fast cooling a monotropic nematic phase was observed at approx 20° C., this indicates that in a mixture containing only compound 1 and compound 2, that compound 2 has a virtual transition of −110° C.

Materials with no significant tendency to undergo LC phase formation will typically depress the N—I transition temperature of a reference material by ˜2° C. for every % added. One would expect an extrapolated clearing point near to −273° C. in such a case, neglecting the effects of phase separation and partition.

Compound 2 is showing an extrapolated N—I of −90.9° C. The results clearly show that a compound which possesses a nitrated mesogenic core and (-1,4-) terminal end groups causes liquid crystalline properties, i.e. the ability to switch between crystalline phases, namely the nematic phase and isotropic phase.

Clearly the compounds of the invention in their primary use as an explosive may not require optimisation of all their LC properties, such as, for example their switching characteristics, i.e. speed of switching. Switching characteristics would only need optimisation if the compounds were to be used in a high refresh rate display.

1.-29. (canceled)

30. A compound of Formula (I),
R₁—(R₁₇—W₁)_{f}—(R₁₈—W₂)_{g}—(R₁₉)—R₂ (I)
where R₁ and R₂ are each independently selected from hydrogen, a functional group, nitro, cyano, halo, optionally substituted hydrocarbyl, optionally substituted alkoxy, optionally substituted heterocyclyl, any of which may be optionally interposed with one or more oxygen or sulphur atoms;
wherein at least one of R₁ and R₂ is selected from a group other than hydrogen or nitro.
R₁₇, R₁₈ and R₁₉ are each independently selected from cycloalkyl, aryl or heterocyclic rings, any of which may be optionally substituted by one or more groups selected from nitro, halo, optionally substituted hydrocarbyl or cyano;
W₁ and W₂ are independently selected from, a direct bond, —C(O)O—, —OC(O)—, —CH₂—, —CH₂CH₂—, —CH₂O—, —OCH₂—, —O—, —S—, —N═N(O)—, —N═N—, —CH═N—, —CH═CH—, or —C≡C—; and where f is 1 or 2, g is 0, 1 or 2, provided that f+g is less than 3, provided that each of R₁₇, R₁₈ and R₁₉, when present, is substituted by one or more nitro groups.

31. The compound according to claim 10, wherein both R₁ and R₂ are selected from a group other than hydrogen or nitro.

32. The compound according to claim 10, wherein each of R₁₇, R₁₈ and R₁₉ is substituted by two or more nitro groups.

33. The compound according to claim 10, wherein W₁ and W₂ are independently selected from, a direct bond, —N═N(O)—, —N═N—, —C(O)O— or —OC(O)—.

34. The compound according to claim 10, wherein R₁₇, R₁₈ and R₁₉ are each phenyl.

35. The compound according to claim 10 wherein each R₁₇, R₁₈ and R₁₉ is an independently selected six membered ring, wherein each ring is linked at positions -1,4- such as to form a linear mesogenic core.

36. The compound according to claim 10 wherein R₁ and R₂ are each independently selected from cyano, halo, branched or a straight chain alkyl, alkoxy, alkenyl, alkenyloxy, alkanoyloxy, alkenoyloxy and are optionally substituted with nitro, nitrate ester or halo.

37. A compound according to claim 10 which is of general Formula (III), 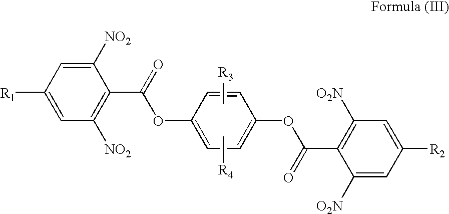
where R₁, R₂ are as defined in claim 10, and R₃ and R₄ are independently selected from hydrogen or nitro.

38. A compound according to claim 10 which is of general Formula (XIX), 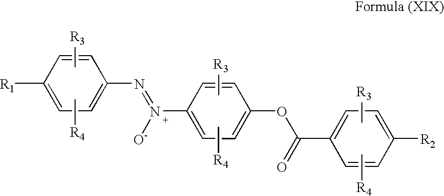
where R₁ and R₂ are as defined in claim 10, and R₃ and R₄ are independently selected from hydrogen or nitro.

39. An explosive composition comprising at least one compound according to claim 10.

40. An explosive composition according to claim 39 which further comprises at least one non-explosive liquid crystal compound or non-explosive liquid crystal mixture.

41. An explosive composition according to claim 40, wherein the at least one non-explosive liquid crystal compound or mixture is present in the range of from 1% to 5% by volume of the explosive composition.

42. An explosive composition according to claim 39 which further comprises a binder present in the amount of from 0.5% to 20%.

43. An explosive composition according to claim 42, wherein the binder is an energetic polymer.

44. An explosive composition according to claim 43, wherein the energetic polymer is selected from Polyglyn (Glycidyl nitrate polymer), GAP (Glycidyl azide polymer) or Polynimmo (3-nitratomethyl-3-methyloxetane polymer).

45. A munition comprising an explosive composition according to claim 39.

46. A munition according to claim 45, comprising an activation means to provide at least one stimulus, which is capable in use of causing a change in the sensitivity of a compound of Formula (I).

47. A munition according to claim 46, wherein the at least one stimulus is an electromagnetic field (EMF), applied electric voltage, induced current, magnetic field or heat source.

48. A munition according to claim 46, adapted such that external heating of the munition will cause a compound of Formula (I), to switch to a less thermally sensitive crystalline state.

49. A liquid crystalline device comprising a compound of formula (I) as defined in claim 30.

50. A method of changing the sensitivity of a compound of Formula (I) according to claim 30, or an explosive composition, comprising the steps of applying at least one stimulus to at least part of said compound or composition.

51. Any process for preparing a compound of Formula (I) as defined in claim 30.

52. A method of forming an explosive, wherein the compound is capable of switching between at least two liquid crystalline states, wherein said explosive comprising a nitrated mesogenic core group, having two terminal end groups attached thereto.

53. A method of forming a high explosive wherein the sensitivity of the explosive is capable of being changed by the application of at least one stimulus, comprising forming a liquid crystalline compound comprising a nitrated mesogenic core group, having two terminal end groups attached thereto.
